(19) United States
(12) Patent Application Publication
(10) Pub. No.: US 2020/0289285 A1
(43) Pub. Date: Sep. 17, 2020

(54) ARTHROPLASTY IMPLANT FOR A FACET JOINT

(71) Applicant: Providence Medical Technology, Inc., Pleasanton, CA (US)

(72) Inventors: Kris Siemionow, Chicago, IL (US); Christopher Lambert, Oakland, CA (US); Shigeru Tanaka, Half Moon Bay, CA (US)

(21) Appl. No.: 16/764,926

(22) PCT Filed: Nov. 16, 2018
(86) PCT No.: PCT/US18/61549

§ 371 Date: May 18, 2020

Related U.S. Application Data
(60) Provisional application No. 62/587,089, filed on Nov. 16, 2017.

(51) Int. Cl. A61F 2/44

(52) U.S. Cl. CPC A61F 2/4405; A61F 2002/3021; A61F 2002/443; A61F 2/4425

(57) ABSTRACT

According to an embodiment, a spinal implant for implantation in a spinal facet joint is disclosed. The spinal implant comprises a first plate having a first surface, a second plate having a second surface, and a biasing element having a first end and a second end, the biasing element coupled to the first surface of the first plate at the first end and the second surface of the second plate at the second end. The biasing element may be at least one of a waveform spring, a coil spring, and a flexible core. The spinal implant may be configured for use in a spinal facet joint, such as a cervical facet joint.

### CROSS REFERENCE TO RELATED APPLICATION

This application claims priority to U.S. Patent Application No. 62/587,089 filed on Nov. 16, 2017, titled Arthroplasty Implant for a Facet Joint, the content of which is incorporated herein in its entirety for any purpose.

### FIELD

This disclosure relates generally to medical devices and methods, and more specifically to devices and methods related to spinal surgery and implants for placement in a facet joint.

### BACKGROUND

The human spine is a segmented series of bones, or vertebrae, separated by cervical discs. Each vertebrae has two facet joints which function to provide stability and guidance during spinal motion. The facet joint comprises two bony surfaces separated by cartilage and surrounded by a capsule of ligaments. Facet joints are susceptible to chronic degenerative diseases, such as facet arthrosis, which result from the degeneration and breaking down of cartilage and bone in the joint structure. Facet arthrosis may affect different areas of the spine, such as cervical facet arthropathy or lumbar facet arthropathy which can result from osteoarthritis and rheumatoid arthritis.

Traditional methods of treatment include non-invasive methods such as rest, changes in sleep position, proper exercise supervised by a physical therapist, and posture correction. More invasive treatments, including spinal fusion, may also be used.

One procedure is spinal arthroplasty, which includes the implantation of a spinal prosthesis to restore posterior element structure and function, as an adjunct to neural decompression. The objective of spinal arthroplasty is to replace all or part of the joint with a prosthesis that stabilizes and decompresses the spine while retaining normal intervertebral motion and mitigating disc degeneration risk.

It is therefore desirable to provide an improved prosthesis that more generally offers improvements or an alternative to existing arrangements.

### SUMMARY

According to an embodiment, a spinal implant, which may also be referred to as an arthroplasty implant, and which may be used for implantation in a spinal facet joint is disclosed. The spinal implant may include a first plate having a first surface, a second plate having a second surface, and a biasing element having a first end and a second end. The biasing element may be coupled to the first surface of first the plate at the first end and the second surface of the second plate at the second end.

In some embodiments, the spinal implant may include a first plurality of teeth coupled to a third surface opposite the first surface of the first plate, and a second plurality of teeth coupled to a fourth surface opposite the second surface of the second plate.

In some embodiments, the first plate may include a first rounded surface opposite to the first surface, and the second plate may include a second rounded surface opposite to the second surface. The first rounded surface and the second rounded surface may be covered in a compliant and high-friction material.

In some embodiments, the spinal implant may include one or more attachment tabs coupled to at least one of the first and second plates.

In some embodiments, the biasing element, may be at least one of a waveform spring, a coil spring, and a flexible core. The flexible core may include at least one of silicone, polyethylene, and hydrogel.

In some embodiments, the spinal implant may include first and second hollow cylinders. The first hollow cylinder may be coupled to the first plate and housing at least a portion of the biasing element. The second hollow cylinder may be coupled to the second plate and housing at least a portion of the biasing element.

In some embodiments, the first plate and the second plate may include a leading edge and a trailing edge, the leading edge configured for insertion into a facet joint. The biasing element may be positioned adjacent to the trailing edge. The spinal implant may include a second biasing element having a third end and a fourth end, the biasing element coupled to the first surface of the first plate at the third end and the second surface of the second plate at the fourth end. The second biasing element may be positioned adjacent to a trailing edge. The leading edge may be tapered.

In some embodiments, the spinal implant may include a central core fixed to one of the first plate and the second plate. The central core may be cylindrical or hemispherical.

In some embodiments, the spinal facet joint may be a cervical spinal facet joint.

According to another embodiment, a spinal implant for implantation in a spinal facet joint is disclosed. The spinal implant may include a top cylindrical portion having a first diameter, a bottom cylindrical portion having a second diameter that is larger than the first diameter and a recess formed therein to receive the top cylindrical portion, a biasing element positioned within the recess between the top cylindrical portion and the bottom cylindrical portion, and a fastener configured to hold the top cylindrical portion within the bottom cylindrical portion and compress the biasing element.

In some embodiments, the fastener may include first and second threaded portions. The first threaded portion may be integrally formed with and extending from the top cylindrical portion. The second threaded portion may be formed in the recess of the bottom cylindrical portion and configured to receive the first threaded portion. The spinal implant may include an engagement feature, such as a notch formed in a side wall of the top cylindrical portion and configured to rotate the top cylindrical portion relative to the bottom cylindrical portion.

In some embodiments, the fastener may include a first hole formed in a side wall of the top cylindrical portion, a second hole formed in a side wall of the bottom cylindrical portion and configured to align with the first hole when the biasing element is compressed, and a set screw configured to be removably inserted into the aligned first and second holes.

In some embodiments, the biasing element may be aligned along a longitudinal axis of the top cylindrical portion and the bottom cylindrical portion.

In some embodiments, the spinal facet joint may be a cervical spinal facet joint.

An arthroplasty implant is disclosed. The implant may include a first facet joint engagement plate having a first surface, a second facet joint engagement plate having a second surface, and a biasing element having a first portion and a second portion, the biasing element coupled to the first surface of the first engagement plate at or near the first portion and the second surface of the second engagement plate at or near the second portion.

In some aspects, the first and second facet joint engagement plates are cervical facet joint engagement plates. In some aspects, the biasing element may be at least one of a waveform spring, a coil spring, and a flexible core. In some aspects, the flexible core includes at least one of silicone, polyethylene, and hydrogel.

An arthroplasty implant is disclosed. In some aspects, the implant includes a first articulating subchondral engagement plate having a first surface, a second articulating subchondral engagement plate having a second surface, and a biasing element having a first portion and a second portion, the biasing element coupled to the first surface of the first engagement plate at or near the first portion and the second surface of the second engagement plate at or near the second portion. In some aspects, the first and second articulating subchondral engagement plates are configured for a cervical facet joint. In some aspects, the biasing element is at least one of a waveform spring, a coil spring, and a flexible core. In some aspects, the flexible core includes at least one of silicone, polyethylene, and hydrogel.

Additional embodiments and features are set forth in part in the description that follows, and will become apparent to those skilled in the art upon examination of the specification or may be learned by the practice of the disclosed subject matter. A further understanding of the nature and advantages of the present disclosure may be realized by reference to the remaining portions of the specification and drawings, which form part of the disclosure. One of skill in the art will understand that each of the various aspects and features of the disclosure may advantageously be used separately in some instances, or in combination with other aspects and features of the disclosure in other instances.

### BRIEF DESCRIPTION OF THE DRAWINGS

The accompanying drawings, which are incorporated into and constitute a part of the specification, illustrate embodiments of the disclosure and, together with the general description above and the detailed description below, serve to explain the principles of these embodiments.

FIG. 1 is a side view of an arthroplasty implant with engagement plates and a biasing element in accordance with aspects of the present disclosure.

FIG. 2 is a side view of the arthroplasty implant of FIG. 1, except the engagement plates are a different size.

FIG. 3 is a perspective view of the arthroplasty implant of FIG. 1 positioned within a facet joint.

FIG. 4 is a side view of an arthroplasty implant with engagement plates and a biasing element positioned within a housing.

FIG. 5 is a sectional view of the arthroplasty implant of FIG. 4.

FIG. 6 is another sectional view of the arthroplasty implant of FIG. 4.

FIG. 7 is a side view of an arthroplasty implant having tapered engagement plates and two biasing elements.

FIG. 8 is a side view of the arthroplasty implant of FIG. 7, except the implant has one biasing element.

FIG. 9 is a perspective view of the arthroplasty implant of FIG. 7 positioned within a facet joint.

FIG. 10A is a side view of an arthroplasty implant having engagement plates and a core of compliant material.

FIG. 10B is a side view of an arthroplasty implant having engagement plates and a core of compliant material.

FIG. 11 is a perspective view of the arthroplasty implant of FIG. 10A positioned within a facet joint.

FIG. 12 is a side view of an arthroplasty implant having engagement plates and a waveform biasing element and a cylindrical central core.

FIG. 13 is a side view of an arthroplasty implant having engagement plates and a waveform biasing element and a cylindrical central core.

FIG. 14 is a side view of an arthroplasty implant having engagement plates and a hemispherical central core.

FIG. 15 is a side view of an arthroplasty implant having engagement plates and a hemispherical central core.

FIG. 16 is a sectional view of the arthroplasty implant of FIG. 15.

FIG. 17A is a perspective view of an arthroplasty implant according to aspects of the present disclosure.

FIG. 17B is a perspective view of an arthroplasty implant according to aspects of the present disclosure.

FIG. 17C is a perspective view of an arthroplasty implant according to aspects of the present disclosure.

FIG. 18A is a side view of the arthroplasty implant of FIG. 17A.

FIG. 18B is a side view of the arthroplasty implant of FIG. 17B.

FIG. 19A is an exploded view of the arthroplasty implant of FIG. 17A.

FIG. 19B is an exploded view of the arthroplasty implant of FIG. 17B.

FIG. 20 is a sectional view of the arthroplasty implant of FIG. 17A, taken along line 20A-20A.

FIG. 21A is a sectional view of the arthroplasty implant of FIG. 17A, taken along line 20A-20A, in a compressed position.

FIG. 21B is a sectional view of the arthroplasty implant of FIG. 17B, taken along line 21B-21B, in a compressed position.

FIG. 22A is a sectional view of the arthroplasty implant of FIG. 17A, taken along line 20A-20A, in an expanded position.

FIG. 22B is a sectional view of the arthroplasty implant of FIG. 17B, taken along line 21B-21B, in an expanded position

FIGS. 23-28 are perspective views of the insertion and release of the implant of FIG. 17A, wherein some or all delivery tool(s) are hidden for clarity.

FIG. 29 is a perspective view of an arthroplasty implant according to aspects of the present disclosure.

FIG. 30 is a sectional view of the arthroplasty implant of FIG. 29 in an expanded position taken along the line 30-30.

FIG. 31 is a perspective view of the arthroplasty implant of FIG. 29 in an expanded position.

FIG. 32 is a sectional view of the arthroplasty implant of FIG. 29 in a compressed position taken along the line 30-30.

FIG. 33 is a perspective view of the arthroplasty implant of FIG. 29 positioned in a facet joint in a compressed position.

FIG. 34 is a perspective view showing the set screw of the arthroplasty implant of FIG. 29 being removed.

FIG. 35 is a perspective view showing the set screw of the arthroplasty implant of FIG. 29 being removed.

FIG. 36 is a side view of an arthroplasty implant having facet joint surface engaging plates and attachment tabs according to aspects of the present disclosure.

FIG. 37 is a sectional view of the arthroplasty implant of FIG. 36.

FIG. 38 is a side view of the arthroplasty implant of FIG. 36, except the engagement plates are a different size.

FIG. 39 is a side view of the arthroplasty implant of FIG. 36 with the attachment tabs positioned at an angle.

FIG. 40 is a sectional view of the arthroplasty implant of FIG. 39.

FIG. 41 is a side view of the arthroplasty implant of FIG. 39, except the engagement plates are a different size.

FIG. 42 is a side view of the arthroplasty implant of FIG. 36 with the attachment tabs positioned at right angles relative to the plates.

FIG. 43 is a sectional view of the arthroplasty implant of FIG. 42.

FIG. 44 is a side view of the arthroplasty implant of FIG. 42, except the engagement plates are a different size.

FIG. 45 is a side view of the arthroplasty implant of FIG. 38 with fasteners.

FIG. 46 is a side view of the arthroplasty implant of FIG. 38 with fasteners, except the central core is removed.

FIG. 47 is a perspective view of the arthroplasty implant of FIG. 46 positioned in a facet joint and secured with fasteners.

FIG. 48 is another perspective view of the arthroplasty implant of FIG. 46 positioned in a facet joint and secured with fasteners.

FIG. 49 is a perspective view of an arthroplasty implant having rounded surfaces according to aspects of the present disclosure.

FIG. 50 is a side view of the arthroplasty implant of FIG. 49.

FIG. 51 is a perspective view of the arthroplasty implant of FIG. 49 positioned in a facet joint.

FIG. 52 is a side view of an arthroplasty implant according to aspects of the present disclosure.

FIG. 53 is a sectional view of the arthroplasty implant of FIG. 52 along line 53-53.

FIG. 54 is a side view of an arthroplasty implant according to aspects of the present disclosure.

FIG. 55 is a side view of an arthroplasty implant according to aspects of the present disclosure.

FIG. 56 is an exploded view of the arthroplasty implant of FIG. 55.

FIG. 57 is a perspective view of an arthroplasty implant according to aspects of the present disclosure.

FIG. 58 is a rear view of the arthroplasty implant of FIG. 57 shown in a first compressed position.

FIG. 59 is a rear view of the arthroplasty implant of FIG. 57 shown in an alternate compressed position.

FIG. 60 is a side view of an arthroplasty implant according to aspects of the present disclosure.

Additional embodiments and features are set forth in part in the description that follows, and will become apparent to those skilled in the art upon examination of the specification or may be learned by the practice of the disclosed subject matter. A further understanding of the nature and advantages of the present disclosure may be realized by reference to the remaining portions of the specification and drawings, which form part of the disclosure. One of skill in the art will understand that each of the various aspects and features of the disclosure may advantageously be used separately in some instances, or in combination with other aspects and features of the disclosure in other instances.

### DETAILED DESCRIPTION

Embodiments described in the present disclosure recognize that previous surgical treatments for degenerative diseases of the facet joint, such as facet arthrosis, often require intrusive surgery such as spinal fusion, or implantation of large prostheses such as a facet replacement system. Further, traditional treatments typically use anterior approaches, i.e., surgical procedures with an incision on the front of the body. Such procedures require surgical precision in order to avoid damaging nerves or arteries between the incision and the facet joint. Finally, most, if not all, arthroplasty implants are specially designed for use in the lumbar spine, which often requires invasive surgery. Disclosed herein are various arthroplasty implants, which may also be referred to as spinal implants, for use in cervical facet joints which are smaller in size and may have different requirements than an implant for the lumbar spine. Examples of the different requirements for lumbar applications include higher expected loads and more difficult anatomical access. The disclosed arthroplasty implants may be implanted using minimally invasive surgery. Additionally, the arthroplasty implants may be inserted using a posterior approach which allows for a more direct path to insertion of the implant in the facet joint. Once implanted, the disclosed embodiments allow the implant to articulate in directions that follow the natural motion of the facet joint.

In various aspects described herein, the arthroplasty implant includes facet joint surface engaging plates and at least one spring or spring-like material positioned between the joint surface engaging plates. In some embodiments, the spring or spring-like material may also be referred to as a biasing element. More specifically, the facet joint surface engaging plates include a superior facet joint surface engaging plate and an inferior facet joint surface engaging plate. The facet joint may be a cervical facet joint (e.g. a joint located between cervical vertebrae) in a human.

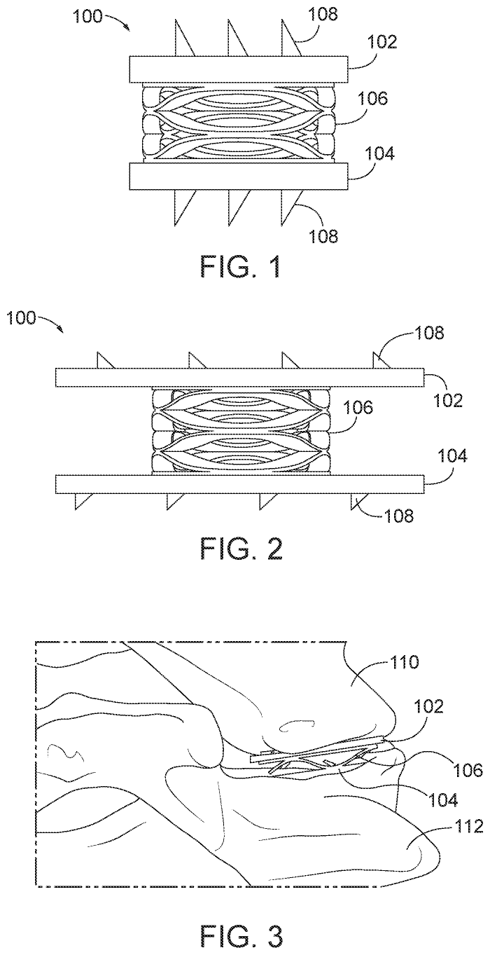

Referring now to FIGS. 1-3, FIG. 1 is a side view of an arthroplasty implant, generally designated 100 with a waveform or disc spring type of biasing element. The implant 100 includes facet joint surface engaging plates including a first or top plate 102, a second or bottom plate 104, and at least one biasing element 106, such as a wave-form or disc spring, and may include a plurality of teeth 108. The top plate 102 may be a superior facet joint surface engaging plate and the bottom plate 104 may be an inferior facet joint surface engaging plate. The plates 102, 104 may generally be made of any suitable rigid material. For example, the top plate 102 and bottom plate 104 may be made of metal (e.g., implant-grade titanium), plastic, a rigid polymer, a combination thereof, or any other suitable material. The biasing element 106, such as a wave-form spring 106, is a disc spring having a frusto-conical shape that provides the spring characteristics and may be made of any material (such as Nitinol) which allows for flexion that mimics the range and directions of motion typical of a facet joint. For example, in one embodiment, the biasing element 106, such as a wave-form spring 106, allows for about 19 degrees of flexion, 14 degrees of extension, 28 degrees of lateral bending, and 17 degrees of rotation. The biasing element 106 may be made, for example, from metal, plastic, rubber, or any other flexible material or polymer. In some embodiments, the biasing element 106 may include a plurality of individual wave-form springs stacked together between the top plate 102 and the bottom plate 104. In various embodiments, the biasing element 106 may include a plurality of welded wave washers to form a generally cylindrical shape. By increasing the number of wave-form springs in the stack, the implant 100 may increase the separation between the upper and lower surfaces of the facet joint as may be desirable for increased movement and support during spine flexion, extension, lateral flexion, compression, rotation and translation.

Each of the top plate 102 and bottom plate 104 may have a plurality of engagement features, such as teeth 108, for fixing or securing the implant 100 in place within the facet joint. The teeth 108 may be coupled or attached to or integrally formed with the top plate 102 and the bottom plate 104. The teeth 108 may be formed of rigid materials such as those used to form the top plate 102 and the bottom plate 104. In various embodiments, the teeth 108 may have a sloped leading edge to facilitate insertion of the implant 100 into the facet joint, and a trailing edge that is substantially perpendicular to the top plate 102 and/or the bottom plate 104, to hold the implant 100 in place within the facet joint.

The top plate 102 and the bottom plate 104 may be positioned and attached to opposing ends or faces of the biasing element 106 or the structure created by welding a plurality of the disc springs 106 together. The top plate 102 and the bottom plate 104 may be substantially parallel when no force is applied to either plate. In the embodiment of FIG. 1, the top plate 102 and the bottom plate 104 may each be cylindrical and have diameters that are substantially the same as the biasing element 106. The top plate 102 and the bottom plate 104 may move relative to each other when the biasing element 106 flexes, extends, compresses, and/or rotates. The pluralities of teeth 108 may be attached to the top plate 102 and the bottom plate 104 opposite to the biasing element 106. By including a plurality of teeth 108 on each of the top plate 102 and the bottom plate 104, the implant 100 may be securely positioned within the facet joint to prevent the implant 100 from disengaging with the facet joint surfaces or otherwise moving within coming out of position from within the joint once set in the desired location by a practitioner. Although shown as triangular engagement features in FIGS. 1 and 2, those skilled in the art will appreciate that the teeth 108 may have any shape suitable for securing or holding the implant 100 in place within the facet joint.

In some aspects, the surface engaging plates may have an increased diameter relative to the biasing element located therebetween. FIG. 2 is a side view of the arthroplasty implant 100 with enlarged diameter top and bottom plates. As shown in the embodiment of FIG. 2, the top plate 102 and the bottom plate 104 may each have a diameter that is substantially greater than the diameter of the biasing element, such as a disc or wave-form spring 106. By increasing the contact area between the top plate 102 and the bottom plate 104 and the respective surface of the facet joint, the implant 100 may be more securely positioned in the facet joint. Additionally, the increased contact area between the top plate 102 and the bottom plate 104 and the facet joint may allow for smaller engagement features 108 (e.g. teeth 108) to help secure the implant 100 in place within the facet joint.

In use, the implant engages the upper and lower surfaces of the facet joint as may be desirable for increased movement and support during spine flexion, extension, lateral flexion, compression, rotation and translation. FIG. 3 is a perspective view of the arthroplasty implant 100 positioned within a facet joint. As shown in FIG. 3, the top plate 102 engages the inferior facet of the superior vertebra 110 of the facet joint, and the bottom plate 104 engages the superior facet of the inferior vertebra 112 of the facet joint. The biasing element 106, such as the wave-form spring 106, applies an expansion force to the top plate 102 and the bottom plate 104 to push against the upper vertebra 110 and the lower vertebra 112, respectively, and hold the implant 100 in place. The teeth 108 (not shown in FIG. 3) provide additional engagement features for holding the implant 100 in place.

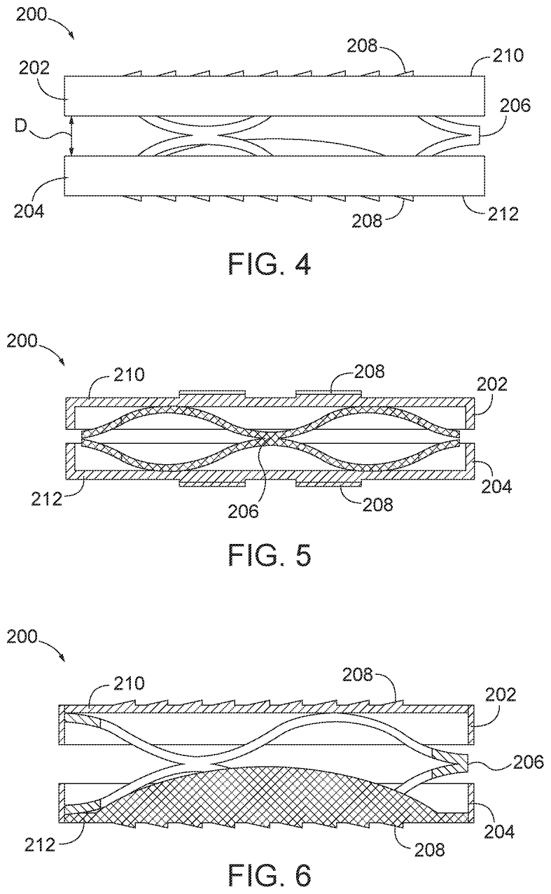

In some aspects, the biasing element may include or be enclosed by a housing for additional structural support. FIG. 4 is a side view of an arthroplasty implant, generally designated 200, with a biasing element, such as a central coil spring enclosed by a housing. The implant 200 generally includes a first or top cylindrical housing 202 having a first or top joint surface engaging plate 210, a second or bottom cylindrical housing 204 having a second or bottom joint surface engaging plate 212, a biasing element 206 (see FIGS. 5 and 6), and a plurality of teeth 208 on at least one of the top or bottom plate. The top housing 202 and the bottom housing 204 may each be made from any suitable rigid material, similar to those listed above with respect to FIG. 1. The top housing 202 and the bottom housing 204 may be aligned along a central axis and separated by a distance, D, which allows for the top housing 202 to flex, extend, compress, and/or rotate relative to the bottom housing 204. Depending on the particular application, the distance, D, may vary between about 0.5 mm and about 2 mm, though other suitable distances are contemplated. The teeth 208 may be made of the same or similar rigid material as the top housing 202 and the bottom housing 204 and may be integrally formed or attached to the top or bottom plate, respectively. The first or top plate may be an engaging plate to the inferior facet of the superior vertebra. The second or bottom plate may be an engaging plate to the superior facet of the inferior vertebra.

FIG. 5 is a sectional view of the arthroplasty implant 200 of FIG. 4 to show the biasing element in more detail. As shown in FIG. 5, the biasing element 206 may be positioned within the top cylinder 202 and the bottom cylinder 204 and attached to the top plate 210 and the bottom plate 212. FIG. 6 is another sectional view of the arthroplasty implant of FIG. 4 and also showing the biasing element within the housing.

The biasing element 206 may generally be any type of biasing element capable of imitating the flexion, extension, contraction, and rotation of a facet joint. In one embodiment, the biasing element 206 is a coil spring or a metal coil spring. In other embodiments, other types of biasing elements and other types of materials may be used. The biasing element 206 is coupled to the top cylinder 202 via the top plate and the bottom cylinder 204 via the bottom plate. For example, one end of the biasing element 206 may be attached to an inside surface of a top plate 210 of the top cylinder 202. The other end of the biasing element 206 may be attached to an inside surface of a bottom plate 212 of the bottom cylinder 204. In use (i.e. when implanted), the biasing element 206 may hold the top cylinder 202 and the bottom cylinder 204 in place relative to each other and separated by the distance, D. Although shown and described as a single, central spring in some embodiments, those skilled in the art will appreciate that the biasing element 206 may comprise more than one biasing element and may be arranged in any suitable configuration to simulate the motion of the facet joint. For example, two, three or four biasing elements may be arranged at regular intervals near the circumference of the inner surfaces of the top cylinder 202 and the bottom cylinder 206. By using multiple biasing elements 206, the motion (e.g., stiffness) of the implant 200 may be tuned by using biasing elements 206 with differing spring constants.

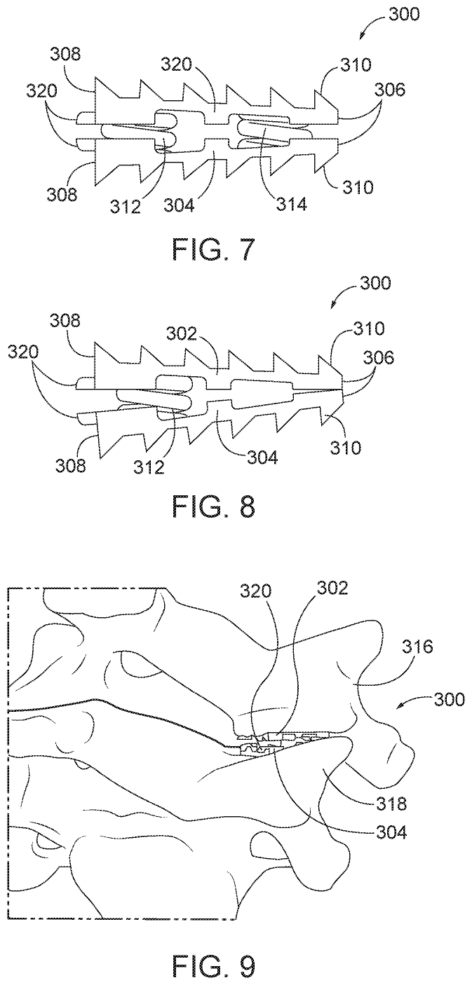

In some aspects, the facet joint surface engaging plates as described herein may have a tapered profile to provide easier entry of the implant into the facet joint. For example, FIG. 7 is a side view of an arthroplasty implant, generally designated 300, having tapered facet joint surface engaging plates and two biasing elements. In the embodiment of FIG. 7, the implant 300 includes a first or top plate 302, a second or bottom plate 304, a first biasing element 312, and a second biasing element 314. Each of the top plate 302 and the bottom plate 304 have a leading end 306 and a trailing end 308. Each of the top plate 302 and the bottom plate 304 may also have a plurality of teeth 310 to fix or secure the implant 300 in position within the facet joint. As discussed above with respect to FIG. 1, the top plate 302, bottom plate 304, and teeth 310 may be formed of any suitable rigid material. The teeth 310 may be integrally formed with or attached or coupled to the top plate 302 and the bottom plate 304.

The top plate 302 and the bottom plate 304 may be coupled together via the first biasing element 312 and the second biasing element 314. The first biasing element 312 may be positioned adjacent to the trailing ends 308, while the second biasing element may be positioned adjacent to the leading ends 306. The first biasing element 312 and the second biasing element 314 may provide an expansion force to the first plate 302 and the second plate 304 to distract the facet joint and also allow for movement of the first plate 302 and second plate 304 that simulates the natural motion of the facet joint. The leading edges 306 of the top plate 302 and the bottom plate 304 may be tapered to facilitate easier insertion of the implant 300 into the facet joint. Additionally, the second biasing element 314 may be compressed during insertion of the implant 300 to make the insertion easier. Each of the trailing ends 308 of the top plate 302 and the bottom plate 304 may include structure (such as a protrusion 320 or recess) configured to engage (e.g., receive) a delivery device, which may be used to insert the implant 300 into the facet joint. For example, a delivery device may engage the protrusions 320 of the top and bottom plates 302, 304 to compress the first biasing element 312 during insertion of the implant 300.

In another embodiment where the facet joint surface engaging plates are tapered, the plates may be separated by a single biasing element. FIG. 8 is a side view of the arthroplasty implant 300 having one biasing element. As shown in FIG. 8, one or both of the biasing elements 312 and 314, may be removed from the implant 300. FIG. 8 shows the implant 300 with the first biasing element 312 located adjacent to the trailing ends 308, but does not include the second biasing element (shown in FIG. 7 as biasing element 314). The embodiment of FIG. 8 may provide for easier insertion of the implant 300 because the leading ends 306 have an increased taper by virtue of the single biasing element configuration and specifically the location of the biasing element near the trailing end of the implant. In use and as shown in FIG. 9, the arthroplasty implant 300 is positioned within a facet joint. The implant 300 may be positioned between an upper vertebra 316 of the facet joint and a lower vertebra 318 of the facet joint. The top plate 302 engages the superior joint surface at the upper vertebra 316 and the bottom plate 304 engages the inferior joint surface at the lower vertebra 318.

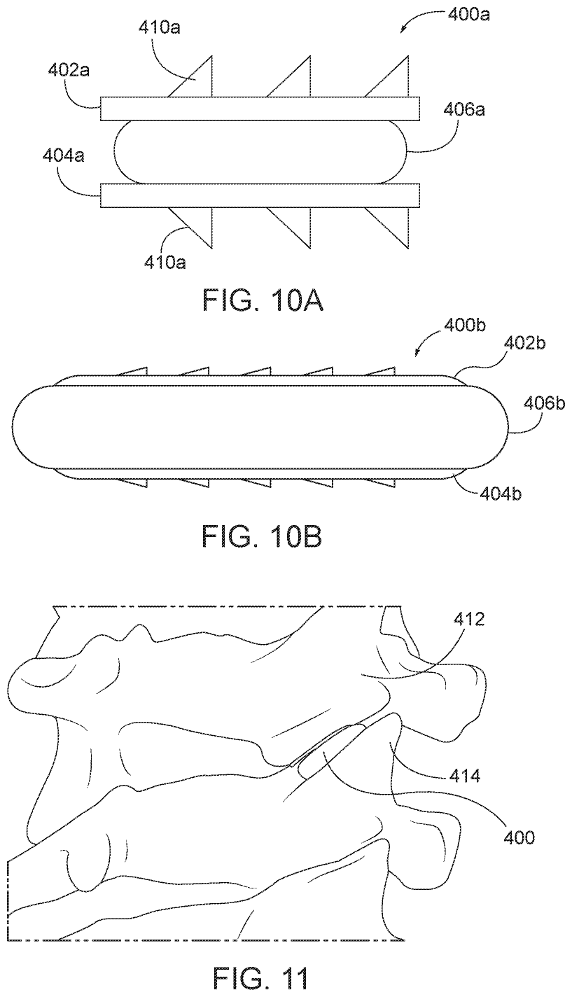

Referring now to FIGS. 10A-11, in some aspects, the facet joint surface engaging plates are coupled together via a spring-like or biased compliant material. FIG. 10A is a side view of an arthroplasty implant, generally designated 400a, having a biasing element of a flexible core of compliant material. The implant 400a generally includes a first or top facet joint surface engaging plate 402a, a second or bottom facet joint surface engaging plate 404a, a biasing element 406a, such as flexible core 406a, and a plurality of teeth 410a. The top plate 402a, bottom plate 404a, and teeth 410a may each be made of similar materials and made in similar ways as the top plate 102, bottom plate 104, and teeth 110 as described above with respect to FIG. 1. The biasing element 406a may be made of a compliant material, such as silicone, polyethylene, or hydrogel. The compliant nature of the biasing element 406a may allow for relative motion between the top plate 402a and the bottom plate 404a in order to simulate the natural motion of the facet joint.

FIG. 10B is a side view of an arthroplasty implant 400b having engagement plates and a biasing element, such as a flexible core of compliant material. The implant 400b of FIG. 10B may be similar to the implant 400a of FIG. 10A with similar components, but with the top plate 402b and the bottom plate 404b being larger or greater in size than the plates 402a, 404a of FIG. 10A. The top plate 402b and the bottom plate 404b may be larger than or have a greater width, length, perimeter and/or other corresponding dimension of the biasing element 406b. When the biasing element is compressed, the top plate 402b and the bottom plate 404b may still be greater in a width, length, perimeter, and/or other corresponding dimension of the compressed biasing element. In some embodiments, the top plate 402b and bottom plate 404b may be similar or dissimilar in size or shape.

In use, and as shown in FIG. 11, the arthroplasty implant 400 of FIG. 10A is positioned within a facet joint. The implant 400 may be positioned between the inferior facet of the superior vertebra 412 of the facet joint and the superior facet of the inferior vertebra 414 of the facet joint. As discussed above, as the facet joint flexes, extends, contracts, and/or rotates, the compliant material may deform while maintaining separation between the upper vertebra 412 and the lower vertebra 414.

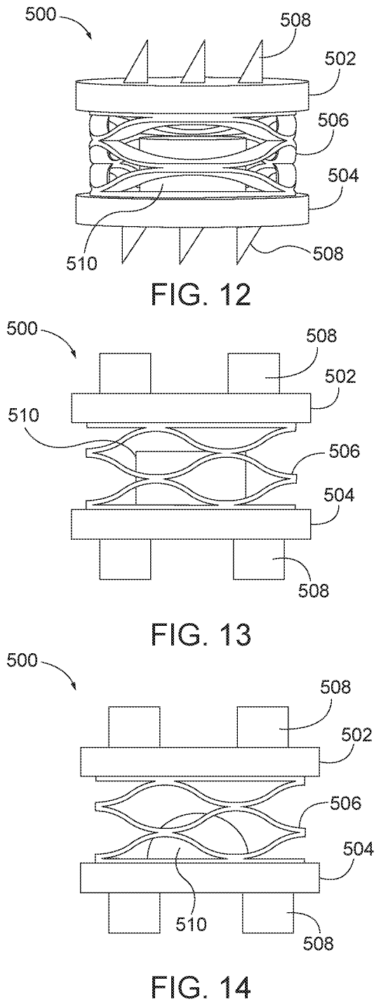

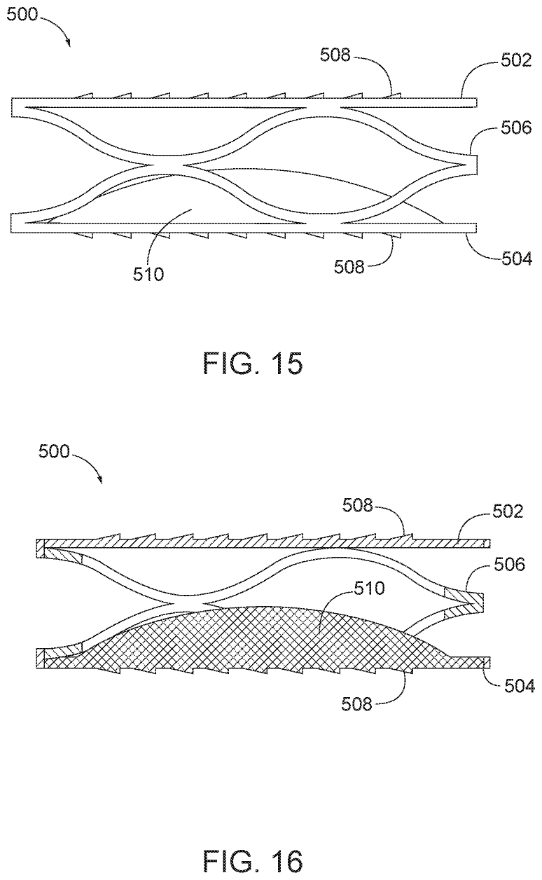

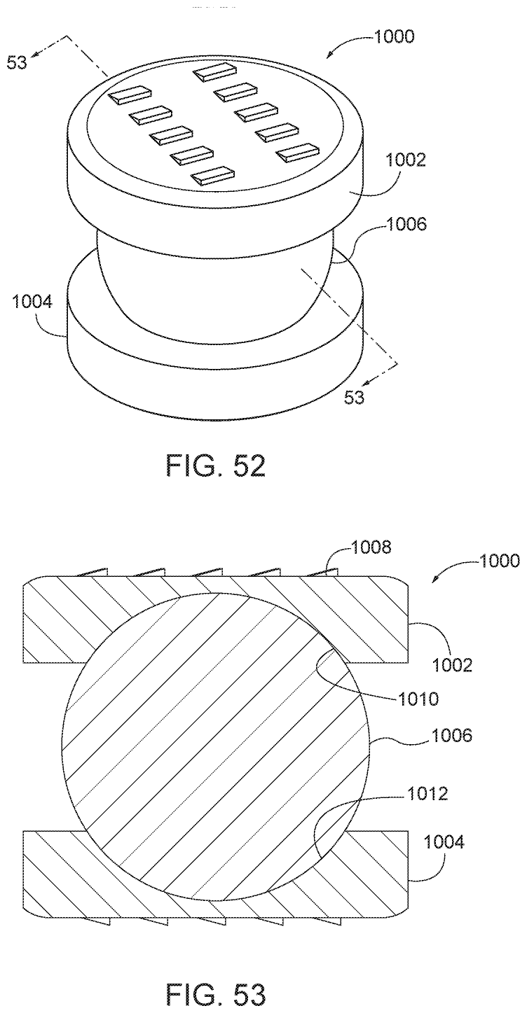

In some aspects, the facet joint surface engaging plates are coupled together via biasing element that may include a spring and a core. FIG. 12 is a perspective view of an arthroplasty implant, generally designated 500, having a biasing element that includes a wave-form spring and a central core. The implant 500 generally includes a first or top plate 502, a second or bottom plate 504, a biasing element 506, a plurality of teeth 508, and a central core 510. Each of the top plate 502, the bottom plate 504, the biasing element 506, and the plurality of teeth 508 may be implemented similarly to the top plate 102, the bottom plate 104, the biasing element 106, and the plurality of teeth 108 as described above with respect to FIG. 1. The central core 510 may be made of a rigid or semi-rigid material such as metal, plastic, hard rubber, or other suitable polymer. The central core 510 may be cylindrical in shape, as shown in FIGS. 12 and 13. Alternatively, the central core 510 may be hemispherical in shape as shown in FIGS. 14 and 15. Other shapes may also be used. The central core 510 may be positioned in the center of and attached or coupled to the bottom plate 504. Alternatively, the central core 510 may be attached or coupled to the top plate 502, such as via mechanical means (e.g., thread, press-fit, fastener) or by adhesive. The central core 510 may limit the flexion, compression, and/or rotation of the top plate 502 relative to the bottom plate 504. For example, as the biasing element is compressed, the top plate may contact the central core 510 before the top plate 502 reaches the bottom plate 504. Accordingly, the amount by which the implant 500 may deform during movement may be limited by the central core 510. The size and the shape of the central core 510 may be determined based on the desired amount of movement. For example, a biasing element that uses a smaller central core, as shown in FIG. 14 may allow for greater range of motion than a comparatively larger central core 510 as shown in FIG. 15. Furthermore, a flat-topped cylindrical core, as in FIGS. 12 and 13, can limit the relative angular motion of the plates 502, 504 better than a spherical or hemi-spherical core, as in FIGS. 14 and 15 and 16. A biasing element that is spherical shaped, such as in FIGS. 52-53, or includes aspherical core, on the other hand, may allow for more complex movement of the plates 502, 504, such as via a combined sliding and tilting motion.

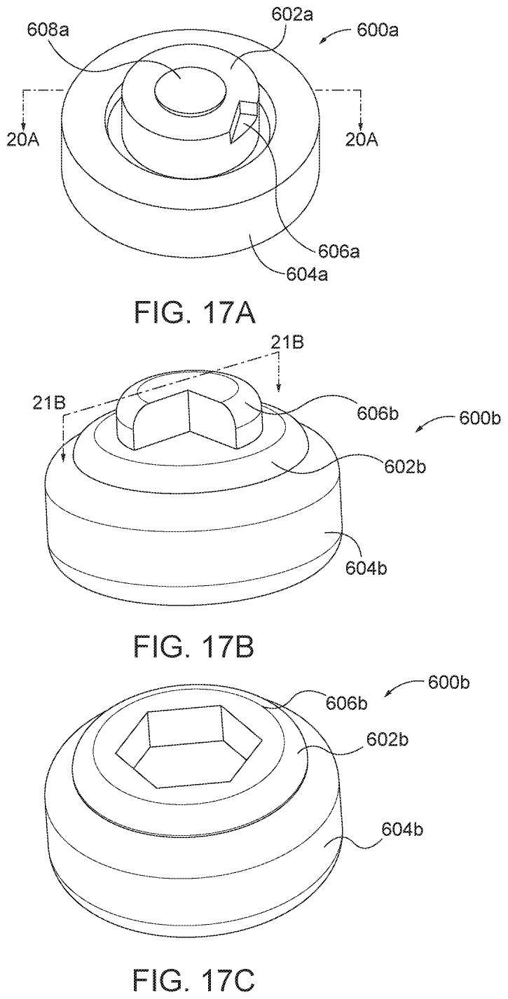

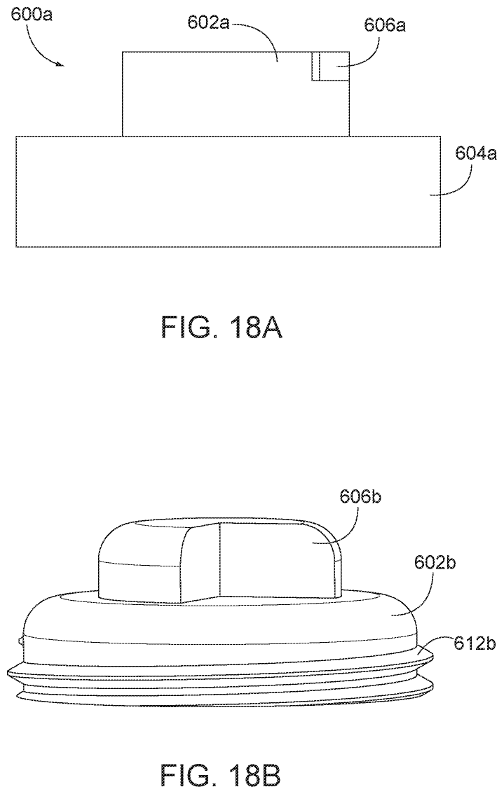

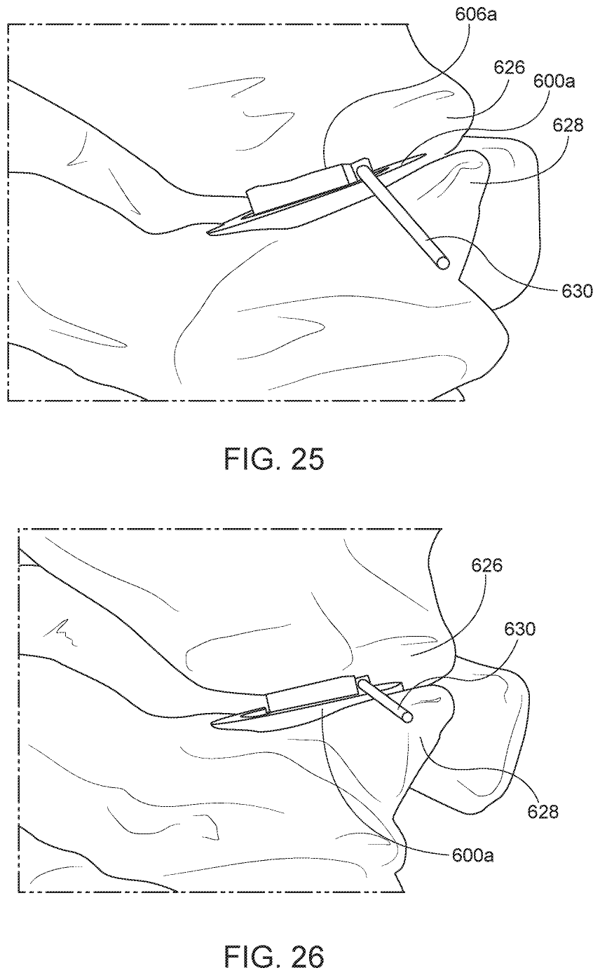

In some aspects, the core or the implant in general may be compressed for insertion into the facet joint. FIG. 17A is a perspective view of an arthroplasty implant, generally designated 600a. FIG. 18A is a side view of the implant 600a. The implant 600a generally includes a first cylinder body 602a, a second cylinder body 604a, and a core 608a. The first cylinder 602a may include an engagement feature 606a, such as a notch, for releasing the first cylinder, as discussed in further detail below with respect to FIG. 25-28. The core 608a may be positioned at least partially within the first cylinder. As shown in FIG. 17A, at least a portion of the first cylinder 602a may be housed within the second cylinder 604a until implanted in the facet joint and allowed to transition from the compressed state to the expanded state.

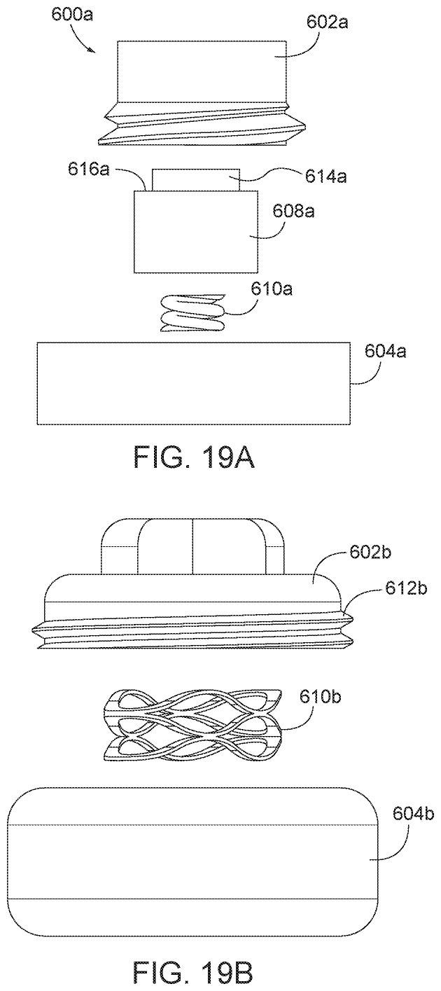

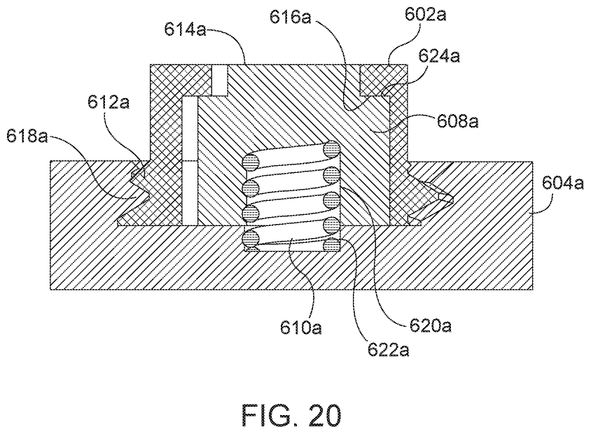

FIG. 19A is an exploded view of the implant 600a. The first cylinder 602a may include a threaded portion 612a at its distal end for securing the first cylinder 602a to the second cylinder 604a, as discussed in further detail with respect to FIGS. 20A, 21A, and 22A. As shown in FIG. 19A, the core 608a may include a cap 614a, which has a diameter that is less than a diameter of the main core body 608a. The cap 614a and core 608a may define a ledge 616a which may be complementary to and configured for receipt at a surface (shown in FIG. 20) within the first cylinder 602a.

The implant 600a may further include a biasing element 610a. The biasing element 610a may be, for example, a coil spring. Other types of biasing elements may also be used. The biasing element 610a may be positioned between the core 608a and the second cylinder 604a. When the implant 600a is compressed, the biasing element 610a may provide an expansion force against the core 608a and the second cylinder 604a. Because the ledge 616a is also in contact with a complementary surface within the first cylinder 602a, the expansion force may also be transferred to the first cylinder 602a.

FIG. 20 is a sectional view of the arthroplasty implant of FIG. 17A, taken along line 20A-20A. As shown in FIG. 20 and discussed above, the first cylinder 602a may have a surface 624a which is complementary to the ledge 616a such that any expansion force exerted by the biasing element 610a on the core 608a is transferred to the first cylinder 602a.

FIG. 20 also shows a first channel 620a defined in the core 608a and configured to receive at least a portion of the biasing element 610a and a second channel 622a defined in the second cylinder 604a and configured to receive at least a portion of the biasing element 610a. The channels may maintain the biasing element in place along a central axis of the core 608a and the second cylinder 622a.

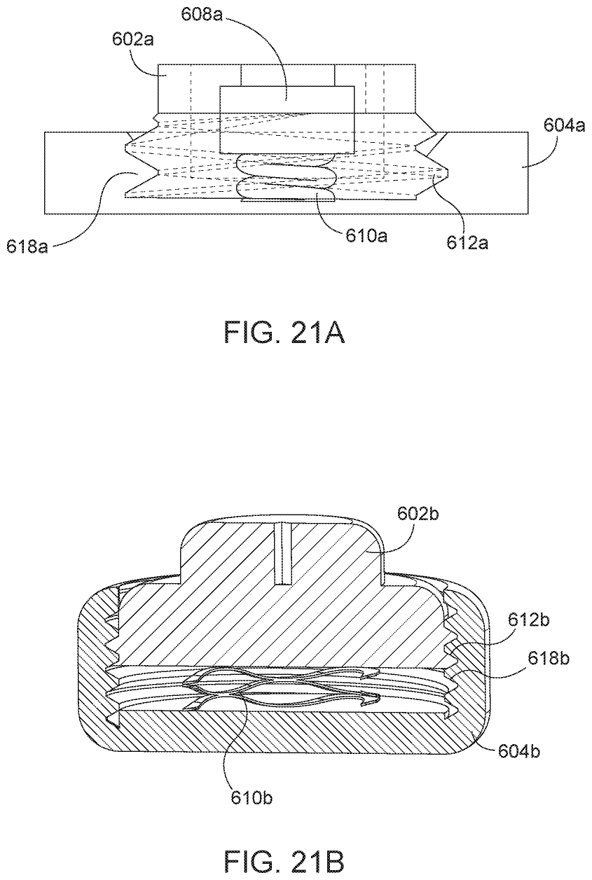

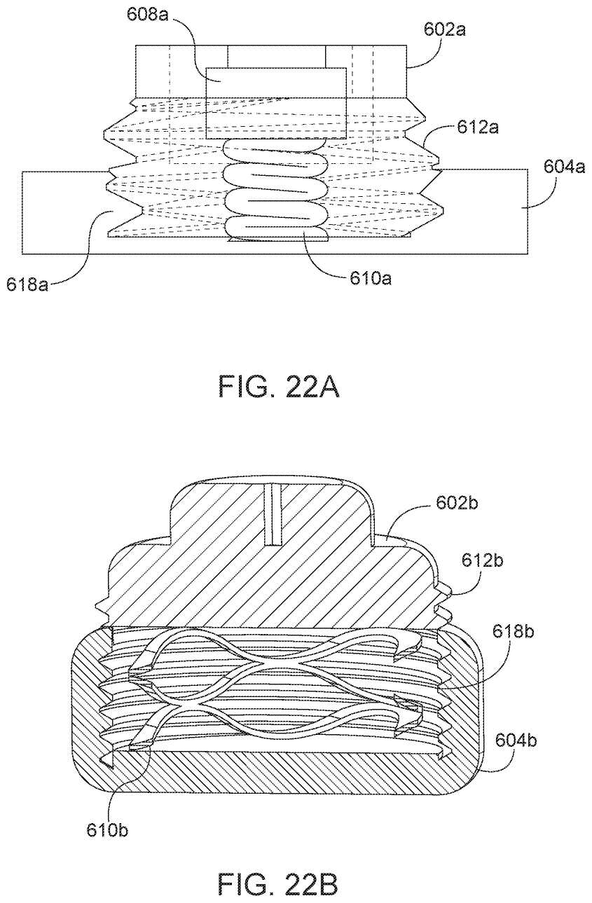

The second cylinder 604a may further include a complementary threaded portion 618a configured to engage the threaded portion 612a of the first cylinder 602a and hold the implant 600a in a compressed state until released after insertion in the facet joint. FIG. 21A is a side view of the arthroplasty implant of FIG. 17A in a compressed position. As shown in FIG. 21A, the threaded portion 612a of the first cylinder 602a engages the threaded portion 618a of the second cylinder 604a to hold the implant 600a in a compressed position. When the implant is in the compressed position, the biasing element 610a may also be in a compressed position. FIG. 22A is a side view of the arthroplasty implant of FIG. 17A in an expanded position. The first cylinder 602a may be rotated relative to the second cylinder 604a to disengage the threaded portions 612a and 618a. Once disengaged, the biasing element 610a may release, applying biasing against the first cylinder 602a and the second cylinder 604a to place the implant 600a in an expanded position.

An additional embodiment of arthroplasty implant 600b is shown in FIGS. 17B, 18B, 19B, 21B, and 22B. Implant 600b may be similar to the implant 600a. An additional embodiment of an arthroplasty implant 600c similar to implant 600b is shown in FIG. 17C. The implants 600b, 600c generally include a first cylinder body 602b, 602c and a second cylinder body 604b, 604c. The first cylinder 602b, 602c may include a feature 606b, 606c for releasing the first cylinder 602b, 602c from the second cylinder 604b, 604c. A difference between the implants 600b, 600c is the feature 606b of FIG. 17B is a raised engagement feature that may include a keyway or notch or other mating device. The feature 606c of FIG. 17C is a recessed engagement feature that may include a keyway or notch or other mating device. In some examples, the keyway or notch may be shaped to receive a hex bit, torx bit, star bit, Philips head, flat head, or other type of keyed tool. In use, the engagement feature may be used to release the first cylinder body from the second cylinder body.

FIG. 19B is an exploded view of the arthroplasty implant 600b of FIG. 17B. FIG. 21B is a sectional view of the arthroplasty implant 600b, taken along line 21B-21B, in a compressed position. FIG. 22B is a sectional view of the arthroplasty implant 600b, taken along line 21B-21B, in an expanded position.

As shown in FIG. 19B, the first cylinder 602b of the implant 600b may include a threaded portion 612b at or adjacent a distal end. The threaded portion 612b may include externally formed or male threads. As shown in FIG. 20B, the second cylinder 604b may also include a threaded portion 618b extending around a portion of the inner circumference of the second cylinder. The threaded portion 618b may include internally formed or female threads. The threaded portions 612b and 618b may be complementary to help releasably secure the first cylinder 602b to the second cylinder 604b.

The implant 600b also includes a biasing element 610b to help adjust the position of the first cylinder 602b with the second cylinder 604b. In some examples, at least one or both of the first cylinder 602b and the second cylinder 604b may include a groove to help position or align the biasing element 610b with respect to the cylinder. This groove does not interfere with the rotation of the first cylinder 602b with the second cylinder 604b.

FIG. 21B shows the implant 600b with the biasing element 610b compressed between the first cylinder 602b and the second cylinder 604b as the threads 612b and 618b are engaged. FIG. 22B shows the implant 600b with the biasing element expanded between the first cylinder 602b and the second cylinder 604b, and were the threads 612b and 618b are no longer engaged. Once the threads 612b, and 618b are disengaged, the first cylinder 602b may be directly disengaged from second cylinder 604b, but still indirectly engaged with the second cylinder 604b via the biasing element 610b.

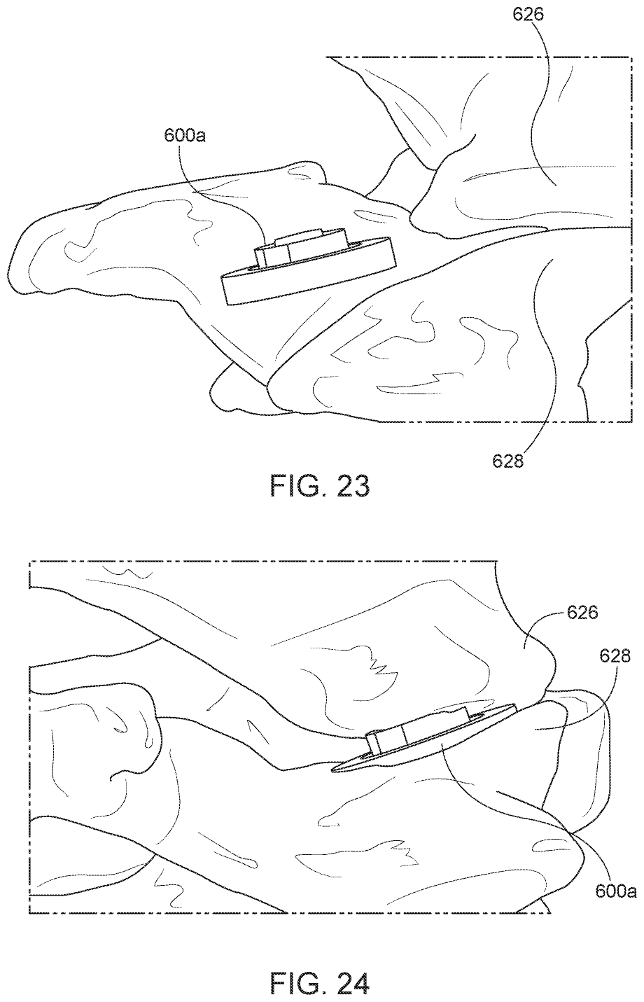

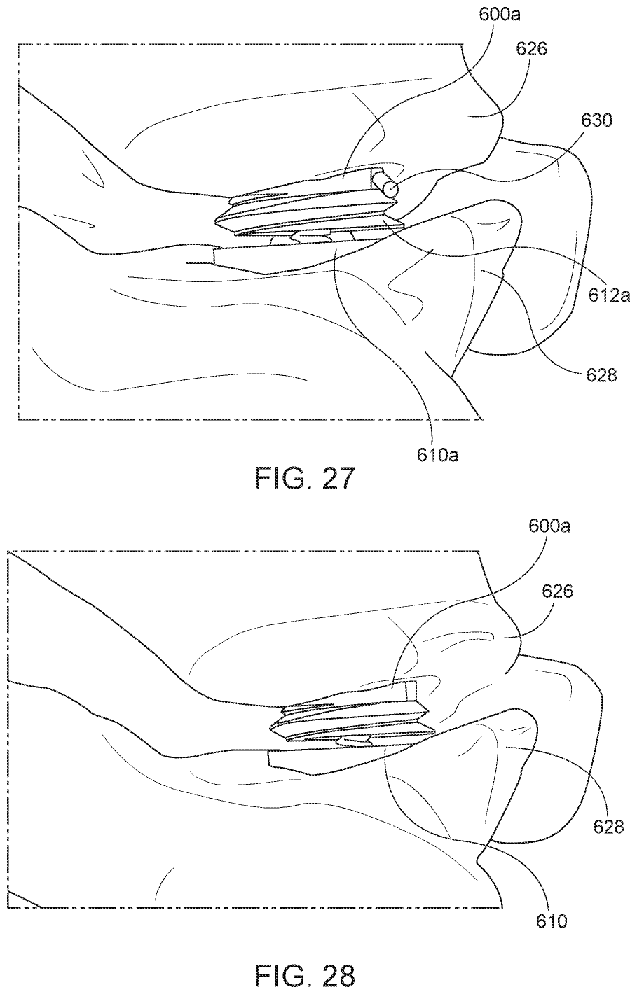

FIGS. 23-28 are perspective views of the insertion and release of the implant 600a of FIG. 17A into a facet joint where some or all of the delivery tools are hidden for clarity. The insertion and release of the implants 600b and 600c into a facet joint may be similar to the insertion and release of the implant 600a. As shown in FIGS. 23 and 24, the implant 600a may be inserted into a facet joint between an upper vertebra 626 and a lower vertebra 628 while in a compressed configuration. For example, the threaded portion 612a of the first cylinder 602a may be engaged with the threaded portion 618a of the second cylinder 604a to hold the biasing element 610a in a compressed position. As shown in FIGS. 25 and 26, a release tool 630, such as a rod or probe, may be used to engage the engagement feature 606a, such as notch 606a, of the implant 600a. The release tool 630 engages the engagement feature causing the first cylinder 602a to rotate relative to the second cylinder 604a and disengage the threaded portion 612a of the first cylinder 602a from the threaded portion 618a of the second cylinder 604a. As shown in FIGS. 27 and 28, once the first cylinder 602a has rotated a sufficient distance relative to the second cylinder 604a, the threaded portion 612a is completely disengaged from the threaded portion 618a, allowing the biasing element 610a to expand and push the first cylinder 602a away from the second cylinder. The implant 600a may then expand to hold it in place within the facet joint between the upper vertebra 626 and the lower vertebra 628. Once the implant 600a is in place and expanded, the release tool 630 may be removed as shown in FIG. 28.

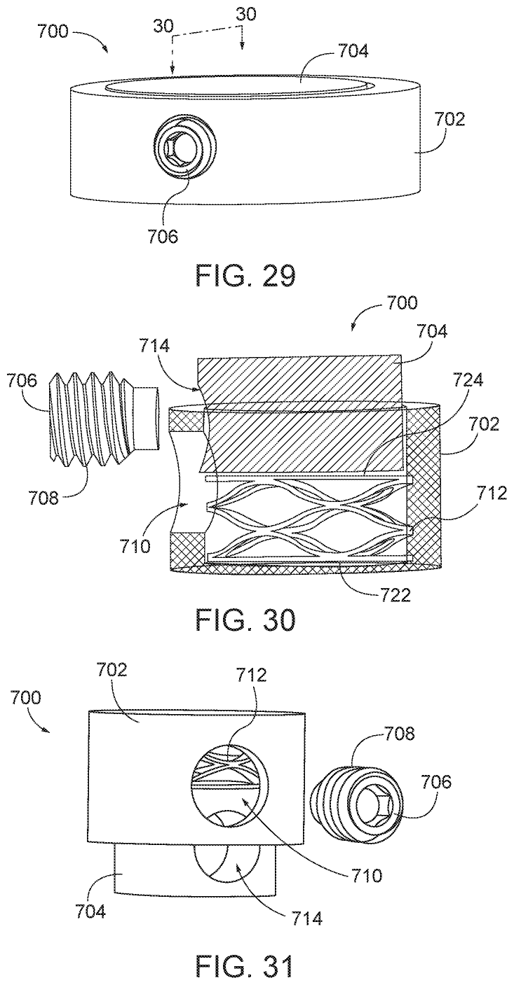

In some aspects, the core and/or the implant may be compressed via a set screw for insertion into the facet joint. FIG. 29 is a perspective view of a arthroplasty implant, generally designated 700. The implant 700 generally includes a bottom cylindrical body 702, a top cylindrical body 704, and a set screw 706. The top cylindrical body and the bottom cylindrical body may each be made of any suitable rigid material as discussed above with respect to FIG. 1. The top cylindrical body 702 may have a diameter that is larger than a diameter of the top cylindrical body 704. The bottom cylindrical body 702 may also have a cavity defined therein and configured to receive the top cylindrical body 704. For example, the cavity may be substantially cylindrical in shape and have a diameter that is sufficiently large to receive the top cylindrical body 704. The set screw 706 may be any type of fastener that is capable of holding the top cylindrical body 704 and the bottom cylindrical body 702 in a compressed position.

FIG. 30 is a sectional view of the arthroplasty implant of FIG. 29 in an expanded position taken along the line 30-30. As shown in FIG. 30, the implant 700 may further include a biasing element 712. The biasing element 712 may be a wave-form spring, as shown in FIG. 30, or any other suitable biasing element, such as coil spring, etc. The biasing element 712 may be positioned within the cavity of the bottom cylindrical body 702 and between a base of the bottom cylindrical body 722 and a base of the top cylindrical body 724. By placing the biasing element 712 between the bottom cylindrical body 702 and the top cylindrical body 704, the biasing element may apply an expansionary force to the top cylindrical body 704 and the bottom cylindrical body 702 causing the implant 700 to expand, which may aid in holding the implant 700 in place within the facet joint.

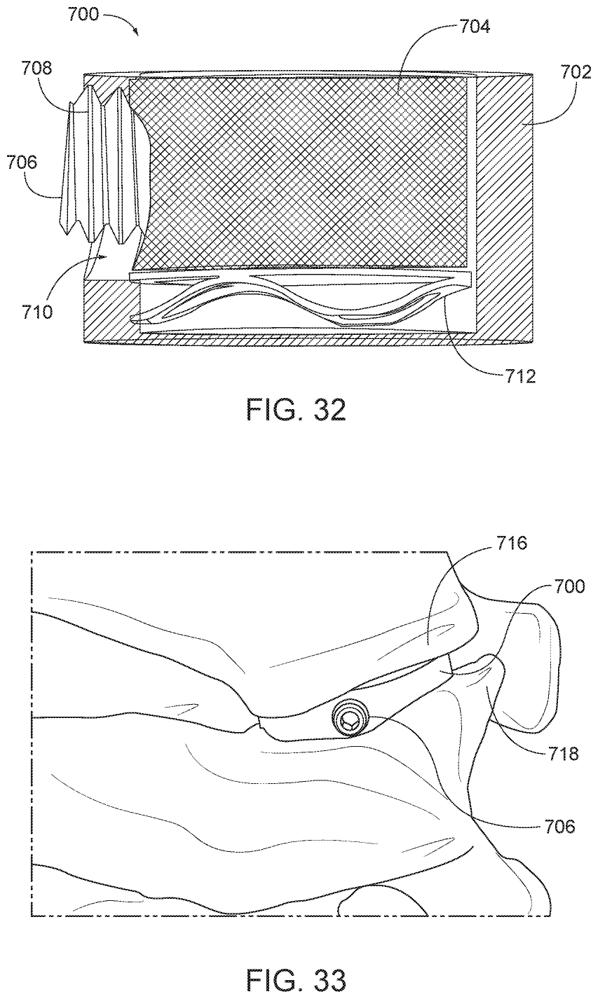

The bottom cylindrical body 702 may have a first hole or aperture 710 formed therethrough and configured to receive the set screw 706. The top cylindrical body 704 may also have a second hole or aperture 714 formed therein and configured to receive the set screw 706. In various embodiments, when the implant 700 is in a compressed position, the first hole 710 and the second hole 714 may align and the set screw 706 may be inserted into both the first hole 710 and the second hole 714 to hold the implant 700 in the compressed position. In some embodiments, the set screw may have a threaded portion 708 to engage a complementary threaded portion in the hole 710 and/or the hole 714. As shown in FIG. 31, when the implant 700 is in an expanded position, the hole 710 and the hole 714 may be misaligned such that the set screw 706 cannot be inserted. When compressed, the hole 710 and the hole 714 align, allowing for insertion of the set screw 706, as shown in FIG. 32.

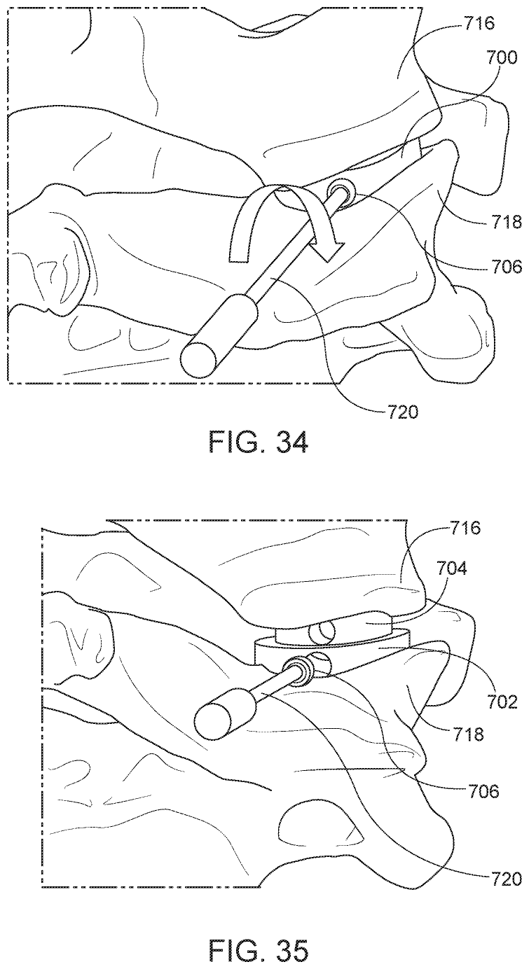

FIG. 33 is a perspective view of the arthroplasty implant 700 positioned in a facet joint in a compressed position. As shown in FIG. 33, the implant 700 may be positioned within a facet joint between an upper vertebra 716 and a lower vertebra 718. FIG. 34 is a perspective view showing the set screw being removed. A release tool 720 may be used to remove the set screw 706 from the implant 700. For example, the set screw 706 may be configured to receive an end of the release tool 720, such as a standard hex key, Philips head screw, or flat head screw. The release tool 720 may engage and rotate the set screw 706 until the screw is completely removed from the implant 700 (see FIG. 35). Once the set screw 706 is disengaged from the implant 700, the release tool 720 and the set screw 706 may be removed and the implant 700 may expand within the facet joint as the biasing element 712 expands.

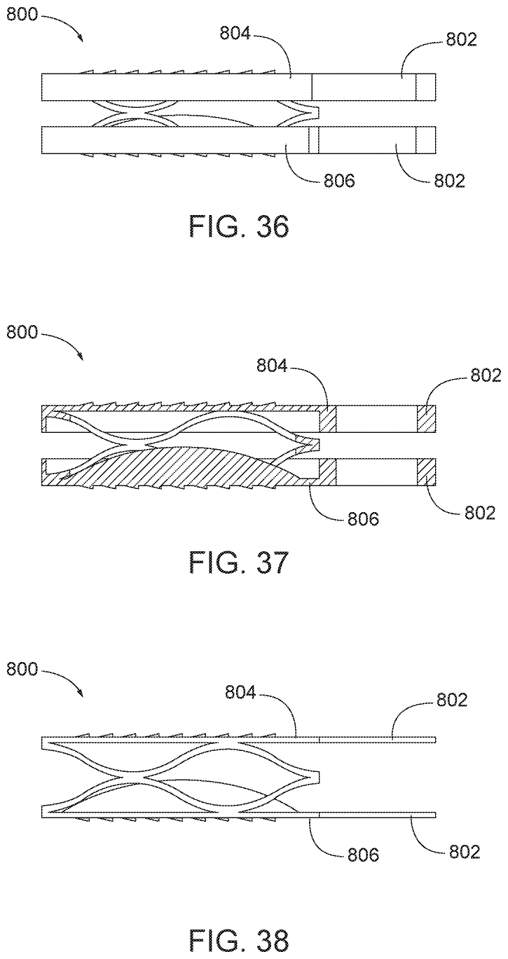

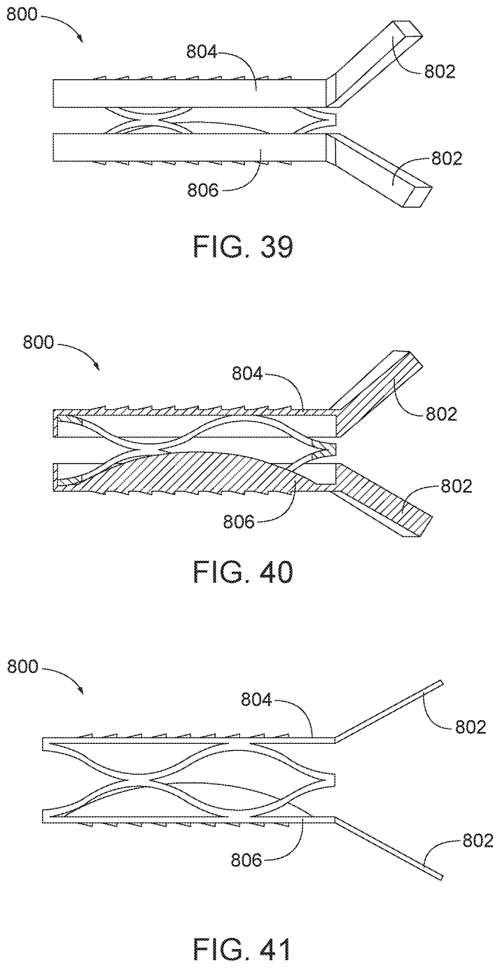

In some aspects, the facet joint engagement plates may include features that contact other surfaces of the vertebrae for fixation of the implant. For example, FIG. 36 is a side view and FIG. 37 is a sectional view of an arthroplasty implant, generally designated 800, having attachment tabs. The implant 800 may include features as described previously such as facet joint surface engagement plates and a biasing element, such as a spring, flexible material or spring-like member as described elsewhere herein positioned therebetween. The implant 800 may include any number of attachment tabs configured to secure the implant to the posterior or lateral edges of the superior and inferior vertebral lateral masses. In the embodiment of FIG. 36, the implant 800 includes a first attachment tab 802 coupled to a first or top facet joint engagement plate 804 and a second attachment tab 802 coupled to a second or bottom facet joint engagement plate 806. The attachment tabs 802 may be made of any suitable material, such as metal, plastic, or other material. The attachment tabs 802 may be fixed in place with respect to the implant 800 or may be hinged, so as to allow for adjustment of the attachment tabs 802 to best secure the implant 800 within the facet joint. As shown in FIG. 36, the attachment tabs 802 may extend substantially parallel to the top plate 804 and the bottom plate 806. FIG. 38 is similar to the embodiment of FIG. 36 except that the tabs 802 and engagement plates 804, 806 are longer and have a smaller width. In FIGS. 39-40, the attachment tabs 802 may be positioned at an angle with respect to the top plate 804 and the bottom plate 806. The angle may be selected so as to make the attachment tabs 802 engage the posterior or lateral edges of the superior and inferior vertebral lateral masses. FIG. 41 is similar to the embodiment of FIGS. 39-40 except that the tabs 802 and engagement plates 804, 806 are longer and have a smaller width.

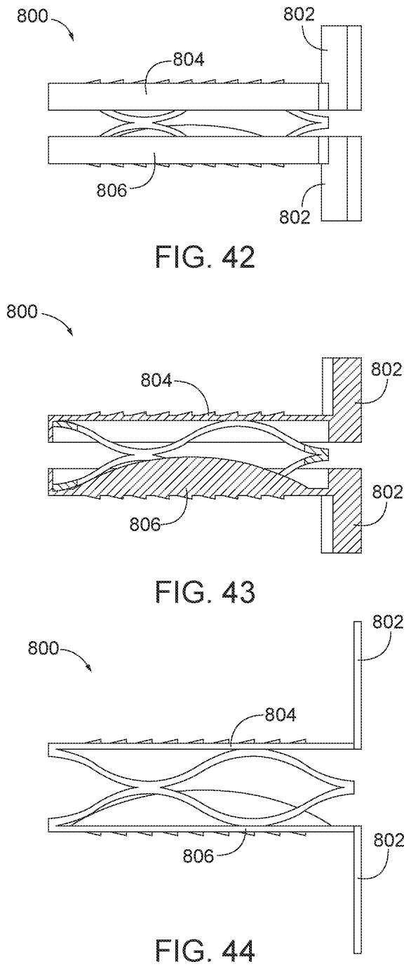

As shown in FIGS. 42-43, the attachment tabs 802 may be positioned substantially perpendicular to the top plate 804 and the bottom plate 806. FIG. 44 is similar to the embodiment of FIGS. 42-43 except that the tabs 802 and engagement plates 804, 806 are longer and have a smaller width.

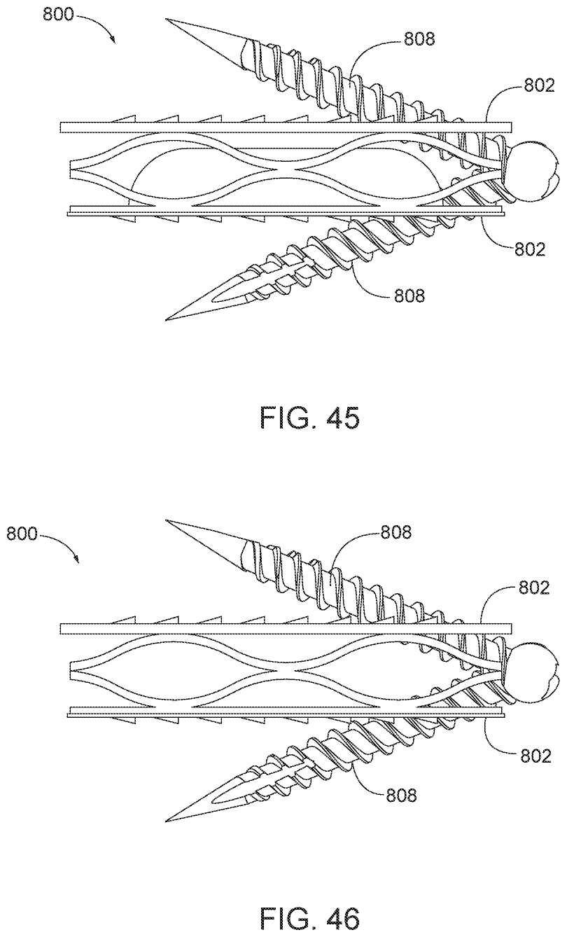

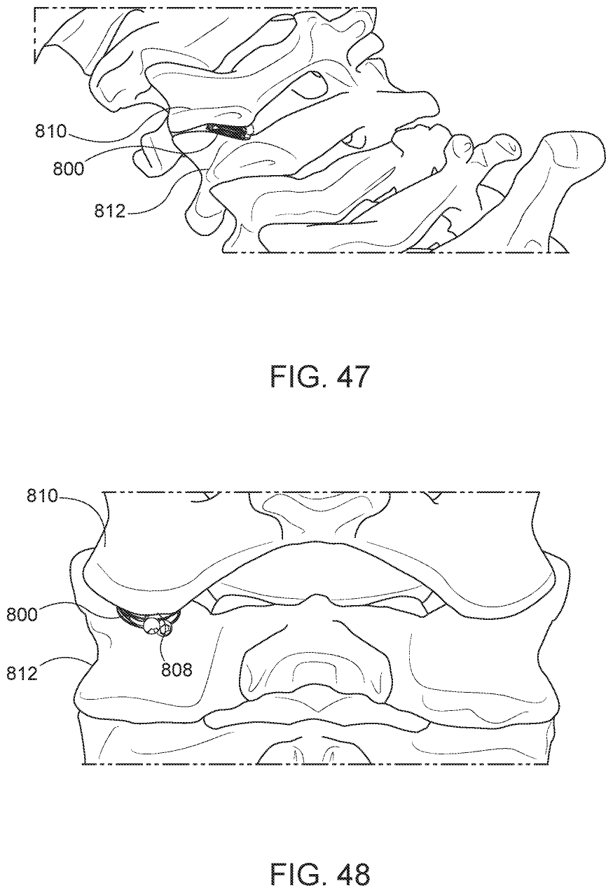

In some aspects, the attachments tabs 802 include openings configured to receive fasteners. FIG. 45 is a side view of the arthroplasty implant 800 with fasteners, such as attachment screws, 808. FIG. 46 is similar to the implant of FIG. 45 except the central or flexible core is removed. The attachment screws 808 may be inserted through openings formed through the attachment tabs 802. The attachment screws 808 may be threaded to secure the attachment screws 808 to the vertebrae. FIGS. 47 and 48 illustrate the implant positioned in a facet joint and fastened with attachment screws. As shown in FIGS. 47-48, one of the attachment screws 808 may secure the implant 800 to the posterior or lateral edges of an upper vertebra 810 of the facet joint, and another attachment screw 808 may secure the implant 800 to the posterior or lateral edges of lower vertebra 812.

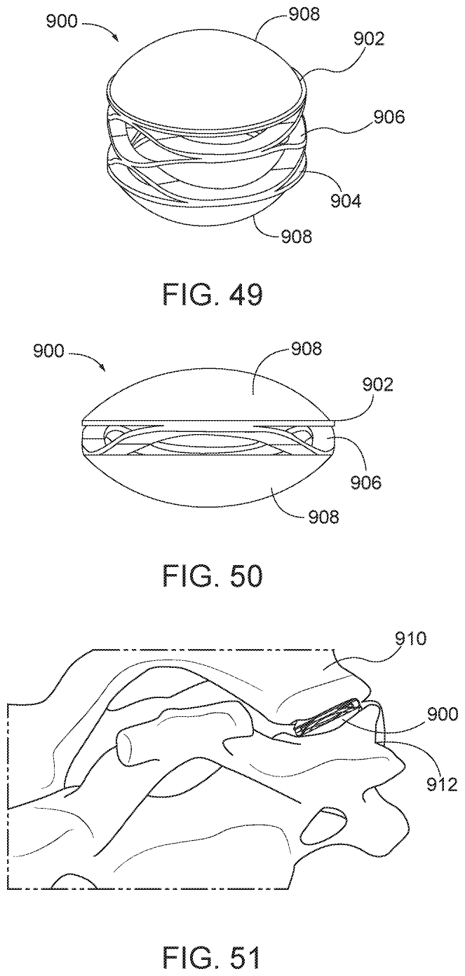

FIGS. 49 and 50 illustrate another fixation mechanism for the implant, generally designated 900 and having rounded surfaces. The implant 900 generally includes a first or top plate 902, a second or bottom plate 904, a biasing element 906 positioned between the top plate 902 and the bottom plate 904, and rounded surfaces 908. Each of the top plate 902, bottom plate 904, and biasing element 906 may be implemented in a similar manner as the top plate 102, bottom plate 104, and biasing element 106 described above with respect to FIG. 1 or elsewhere herein. In some aspects, the rounded surfaces 908 may be covered in a compliant, high-friction material to retain the implant 900 in the facet joint. The rounded surfaces 908 may be positioned on the top plate 902 and the bottom plate 904 opposite the biasing element 906. In some aspects, the rounded surfaces 908 may be rigid and rounded to mimic the profile of the facet joint. As shown in FIG. 51, the implant 900 may be positioned within a facet joint having an upper vertebra 910 and a lower vertebra 912. The rounded surfaces 908 may contact the upper vertebra 910 and the lower vertebra 912 to hold the implant 900 in place within the facet joint.

FIGS. 52 and 53 are views of an arthroplasty implant. FIG. 52 is a side view of an arthroplasty implant 1000. FIG. 53 is a sectional view of the arthroplasty implant 1000 of FIG. 52 along line 53-53. The implant 1000 may be similar to the implant 400 of FIG. 10A. The implant includes a top plate 1002 and a bottom plate 1004, with a biasing element 1006, such as an elastomer or a flexible core, positioned between the plates. Each plate 1002, 1004 may include teeth 1008 extending from an outer surface and that may be used to engage with a vertebra surface. As shown in FIG. 53, the inside surfaces 1010 and 1012 of the top and bottom plates 1002, 1004 may form a shaped surface, such as a concave or convex surface. The biasing element 1006 may be shaped to interact or complement the shaped surfaces 1010, 1012. In some embodiments, the biasing element 1006 may be spherical shaped and the inner surfaces 1010, 1012 are concave shaped. This configuration may allow for the implant 1000 to include a tilting or rolling movement in addition to vertical compression when inserted or used within a facet joint.

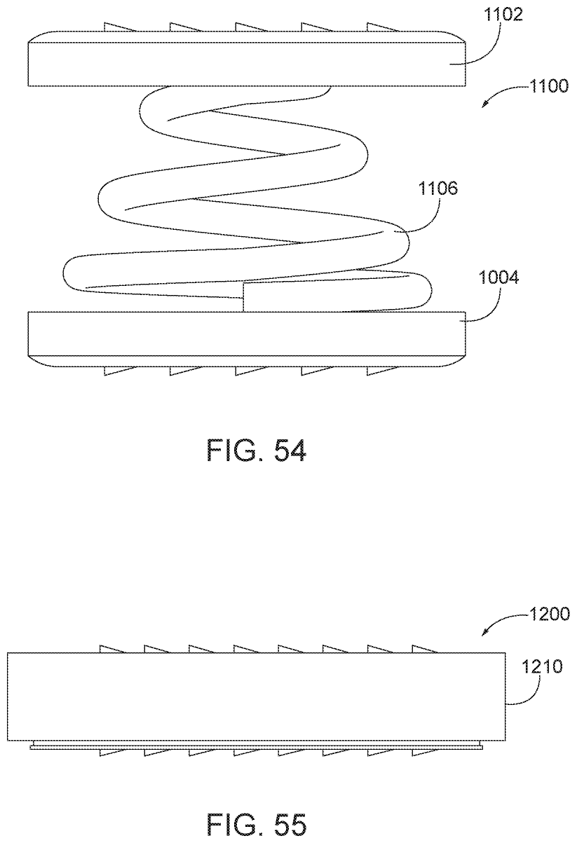

FIG. 54 is a side view of an arthroplasty implant 1100. The implant 1000 may be similar to the implant 200 of FIG. 4. The implant 1100 includes a top plate 1102, a bottom plate 1104, and a biasing element 1106. The biasing element 1106 may be frustum or conical shape, in some examples a coil spring or elastomer, with one end being larger than the other. This shape of the spring may allow for the implant 1100 to include a tilting or rolling movement in addition to vertical compression when inserted or used within a facet joint.

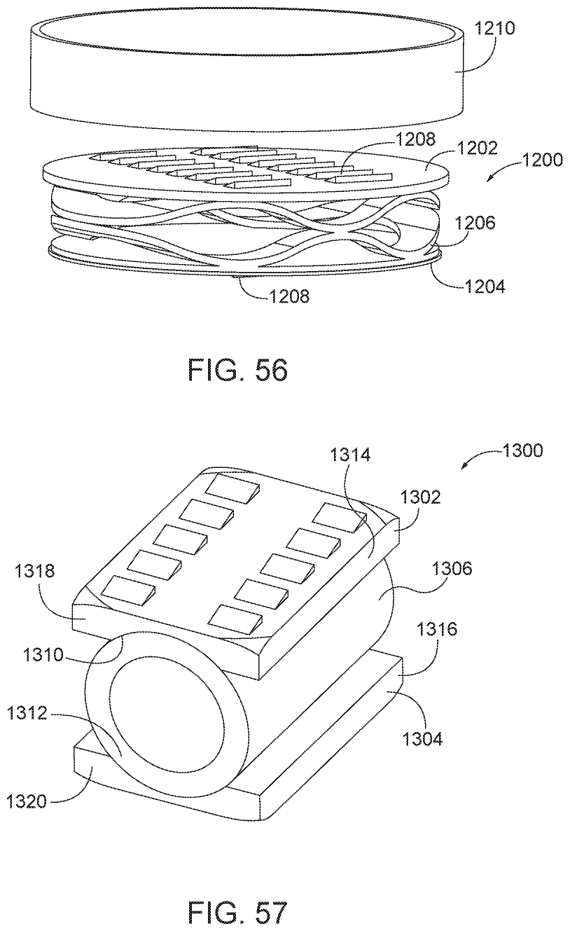

FIG. 55 is a side view of an arthroplasty implant 1200. FIG. 56 is an exploded view of the arthroplasty implant 1200. In some aspects, the implant 1200 may be similar to the implant 100 of FIG. 1. The implant 1200 includes a top plate 1202 with teeth 1208 extending from an outer surface or upper surface, a bottom plate 1204 with teeth 1208 extending from an outer surface or a lower surface, and a biasing element 1206 positioned between the top plate 1202 and the bottom plate 1204. In addition, a sleeve 1210 may extend around an outer perimeter of the assembly of the plates 1202, 1204 and biasing element 1206. The sleeve 1210 may be formed of an elastomer or flexible material with a stiffness that is similar to or greater than a stiffness of the biasing element 1206. The sleeve 1210 may help encapsulate the assembly and provide stability to the implant. In some examples, the sleeve 1210 may help dampen the biasing element 1206 to aid in the implant insertion assembly and in use once the implant is implanted between vertebrae. This may be helpful when the loading on the plates is uneven. Uneven loading may cause the implant to undesirably move or shift positions. Dampening of the biasing element may provide stability for the implant to prevent undesirably movement or shifting.

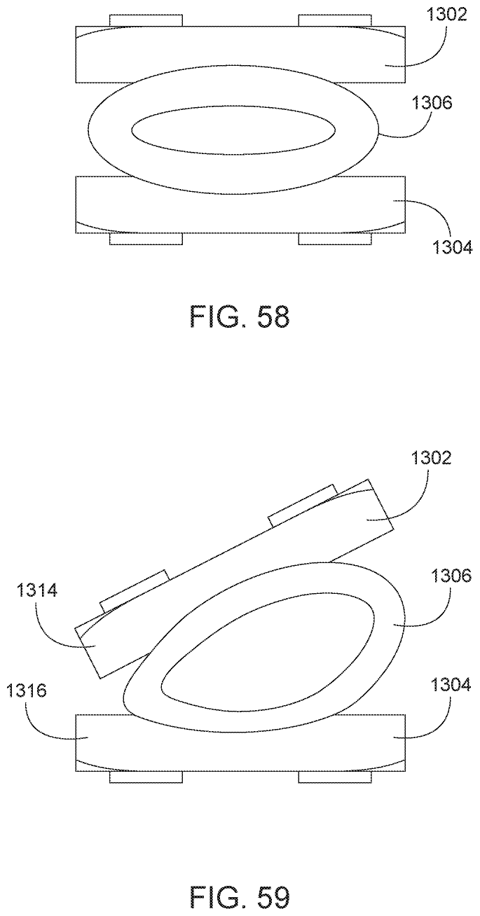

FIGS. 57-59 are views of an arthroplasty implant 1300. FIG. 57 is a perspective view of an arthroplasty implant. FIG. 58 is a rear view of the arthroplasty implant of FIG. 57 shown in a first compressed position. FIG. 59 is a rear view of the arthroplasty implant of FIG. 57 shown in an alternate compressed position. In some aspects, the implant 1300 may be similar to the implant 1000 of FIG. 52. The implant 1300 includes a top plate 1302 and a bottom plate 1304. Similar to implant 1000, the inner surface 1310 and 1312 of the top and bottom plates 1302, 1304 is concave shaped. The implant 1300 also includes a bias element 1306, such as an elastomer. In the implant 1300, the bias element 1306 may be cylindrically shaped, with either solid or hollow central section. The bias element 1306 may be positioned between the top and bottom plates 1302, 1304, with the round outer edges of the bias element 1306 adjacent the concave surfaces 1310, 1312. In addition to vertical compression, the configuration of the implant 1300 may allow the long edges 1314, 1316 of the top and bottom plates 1302, 1304 to rotate towards and away from each other, for example in FIG. 59, about an axis of the bias element 1306. The shape may also allow for the bias element 1306 to be unevenly compressed, for example, where the ends 1318 and 1320 are compressed towards each other, while the ends that are opposite ends 1318, 1320 are compressed to a lesser amount.

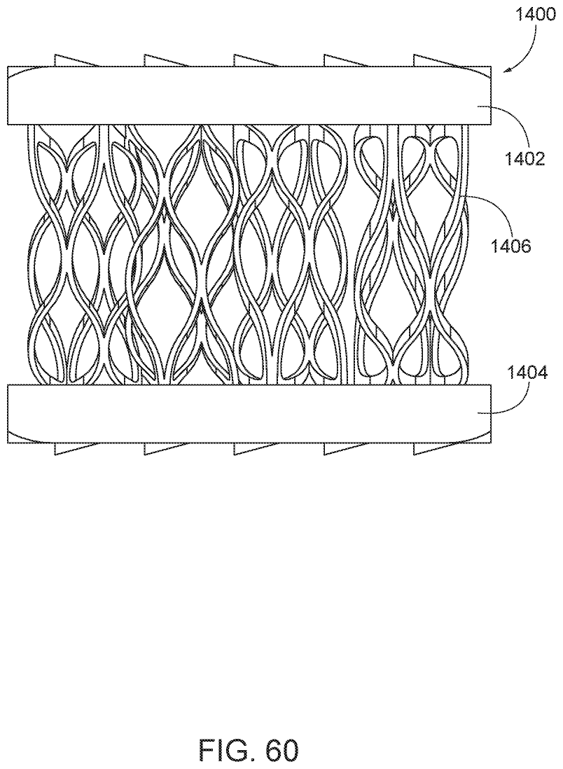

FIG. 60 is a side view of an arthroplasty implant 1400. The implant 1400 of FIG. 60 may be similar in aspect to the implant 1100 of FIG. 54. The implant 1400 includes a top plate 1402, a bottom plate 1404, and a biasing element 1406. In the implant of 1400, the basing element allows for both a vertical compression of the implant, in either an even application or uneven application, and may also allow for a controlled translational movement of the plates 1402, 1404. In some embodiments, the biasing element 1406 may include a vertical wave-form spring, or other biasing element that allows the described controlled movement.

All relative and directional references (including: upper, lower, upward, downward, left, right, leftward, rightward, top, bottom, side, above, below, front, middle, back, vertical, horizontal, and so forth) are given by way of example to aid the reader's understanding of the particular embodiments described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other unless specifically set forth in the claims.

Those skilled in the art will appreciate that the presently disclosed embodiments teach by way of example and not by limitation. Therefore, the matter contained in the above description or shown in the accompanying drawings should be interpreted as illustrative and not in a limiting sense. Thus, it is intended that the scope of the present disclosure should not be limited by the particular embodiments described above.

What is claimed is:

1. A spinal implant comprising:
a first plate having a first surface;
a second plate having a second surface; and
a biasing element having a first end and a second end, the biasing element coupled to the first surface of first plate at the first end and the second surface of the second plate at the second end.

2. The spinal implant of claim 1, further comprising:
a first plurality of teeth coupled to a third surface opposite the first surface of the first plate; and
a second plurality of teeth coupled to a fourth surface opposite the second surface of the second plate.

3. (canceled)

4. (canceled)

5. The spinal implant of claim 1, further comprising:
one or more attachment tabs coupled to at least one of the first and second plates.

6. The spinal implant of claim 1, wherein the biasing element is at least one of a waveform spring, a coil spring, and a flexible core.

7. The spinal implant of claim 6, wherein the flexible core comprises at least one of silicone, polyethylene, and hydrogel.

8. The spinal implant of claim 1 further comprising:
a first hollow cylinder coupled to the first plate and housing at least a portion of the biasing element; and
a second hollow cylinder coupled to the second plate and housing at least a portion of the biasing element.

9. The spinal implant of claim 1, wherein the first plate and the second plate have a leading edge and a trailing edge, the leading edge is tapered configured for insertion into a facet joint.

10. The spinal implant of claim 9, wherein the biasing element is positioned adjacent to the trailing edge.

11. The spinal implant of claim 1, further comprising:
a second biasing element having a third end and a fourth end, the biasing element coupled to the first surface of the first plate at the third end and the second surface of the second plate at the fourth end.

12. The spinal implant of claim 11, wherein the second biasing element is positioned adjacent to a trailing edge.

13. (canceled)

14. The spinal implant of claim 1, further comprising:
a central core fixed to one of the first plate and the second plate.

15. (canceled)

16. The spinal implant of claim 1 any of the preceding claims, wherein the spinal facet joint is a cervical spinal facet joint.

17. A spinal implant comprising:
a top cylindrical portion having a first diameter;
a bottom cylindrical portion having a second diameter that is greater than the first diameter and a recess formed therein to receive the top cylindrical portion;
a biasing element positioned within the recess between the top cylindrical portion and the bottom cylindrical portion; and
a fastener configured to hold the top cylindrical portion within the bottom cylindrical portion and compress the biasing element.

18. The spinal implant of claim 17, wherein the fastener comprises:
a first threaded portion, integrally formed with and extending from the top cylindrical portion; and
a second threaded portion formed in the recess of the bottom cylindrical portion and configured to receive the first threaded portion.

19. The spinal implant of claim 18, further comprising:
an engagement feature formed in a side wall of the top cylindrical portion and configured to rotate the top cylindrical portion relative to the bottom cylindrical portion.

20. The spinal implant of claim 19 wherein the engagement feature is a notch.

21. The spinal implant of claim 17, wherein the fastener comprises:
a first hole formed in a side wall of the top cylindrical portion;
a second hole formed in a side wall of the bottom cylindrical portion and configured to align with the first hole when the biasing element is compressed;
a set screw configured to be removably inserted into the aligned first and second holes.

22. (canceled)

23. (canceled)

24. (canceled)

25. (canceled)

26. (canceled)

27. (canceled)

28. An arthroplasty implant comprising:
a first articulating subchondral engagement plate having a first surface;
a second articulating subchondral engagement plate having a second surface; and
a biasing element having a first portion and a second portion, the biasing element coupled to the first surface of the first engagement plate at or near the first portion and the second surface of the second engagement plate at or near the second portion,
wherein the wherein the first and second articulating subchondral engagement plates are configured for a cervical facet joint.

29. (canceled)

30. The arthroplasty implant of claim 28, wherein the biasing element is at least one of a waveform spring, a coil spring, and a flexible core.

31. The arthroplasty implant of claim 30, wherein the flexible core comprises at least one of silicone, polyethylene, and hydrogel.
